(19) United States
(12) Patent Application Publication
(10) Pub. No.: US 2019/0365878 A1
(43) Pub. Date: Dec. 5, 2019

(54) PHARMACEUTICAL TREATMENTS FOR PREVENTING OR TREATING PANCREATIC CANCER

(71) Applicant: THE JOHNS HOPKINS UNIVERSITY, Baltimore, MD (US)

(72) Inventor: Lei Zheng, Ellicott City, MD (US)

(21) Appl. No.: 16/348,174

(22) PCT Filed: Nov. 6, 2017
(86) PCT No.: PCT/US2017/060157

§ 371 Date: May 8, 2019

Related U.S. Application Data
(60) Provisional application No. 62/418,817, filed on Nov. 8, 2016.

(51) Int. Cl. A61K 39/00; A61K 39/395; A61K 31/404; A61K 31/675

(52) U.S. Cl. CPC A61K 39/0011; A61K 39/3955; A61K 31/404; A61K 31/675; A61K 2039/585; A61K 2039/852; A61K 2039/523; A61K 2039/55522; A61K 2039/5152

(57) ABSTRACT

Described are methods for treating or preventing cancer in a subject by administering a vaccine, an inhibitor of idoleamine 2,3-dioxygenase to the subject. The vaccine may comprise and allogenic PDA tumor cell engineered to secrete granulocyte macrophages colony-stimulating factor (GM-CSF).

### REFERENCE TO RELATED APPLICATIONS

This application claims the benefit of U.S. Provisional Patent Application No. 62/418,817, filed on Nov. 8, 2016, which is hereby incorporated by reference for all purposes as if fully set forth herein.

### BACKGROUND OF THE INVENTION

Cancer immunotherapy is considered to be one of the biggest breakthroughs in cancer therapy in the last decade. However, the success of immunotherapy has so far been limited to a few solid malignancies including melanoma, renal cell carcinoma, non-small-cell lung cancer (NSCLC) and few hematologic malignancies. GVAX is a cancer vaccine composed of whole tumor cells genetically modified to secrete the immune stimulatory cytokine, granulocyte-macrophage colony-stimulating factor (GM-CSF), and then irradiated to prevent further cell division. The product exists as both autologous (patient specific) and allogeneic (non-patient specific) therapy. Results from a phase II clinical trial of resected pancreatic cancer patients showed that GVAX increased the median survival from 15 to 20 months (historical survival rates), to 24.8 months, an increase of more than 25%. In addition, the vaccine increased the one-year survival rate from 63% to 85%, an increase of 35%.

Indoleamine 2,3-dioxygenase (IDO1; IDO2 IDO), including indoleamine 2,3-dioxygenase-1 (IDO1) and indoleamine 2,3-dioxygenase-2 (IDO2) is a heme-containing, monomeric oxidoreductase that catalyzes the degradation of the essential amino acid tryptophan (trp) to N-formyl-kynurenine is encoded by the IDO1(or IDO) gene, which can be subsequently metabolized through a series of steps to form nicotinamide adenine dinucleotide (NAD⁺). The biologic relevance of IDO to peripheral immune tolerance was first proposed when it was shown that treating mice with 1-methyl-tryptophan (1MT), a small-molecule inhibitor of IDO, could break the tolerogenic state that protects allogeneic concepti from the maternal immune system. The role of IDO in immunomodulation, or as an immune checkpoint protein, has since been corroborated in studies with numerous animal models, including models of allograft tolerance, inflammation, and cancer.

Recent interest has focused on the role of IDO in the induction of tumor immune tolerance. It has been shown that tumor cells can evade immune destruction despite displaying recognizable antigens on their surface and the presence of high-avidity T cells that are specific for the antigens. Further, histologic evaluation of most human tumor tissues has shown extensive infiltration by various pro-inflammatory and immune cells, suggesting that the immune system responds to malignancy, but the response is nevertheless ineffective in eliminating tumor cells in most cases. These observations have led to the hypothesis that dominant mechanisms of immune suppression may be responsible for the inability of the immune system to effectively respond to tumor-associated antigens in a way that consistently results in tumor rejection. Previous studies suggest that IDO may be an important regulator of the immunosuppressive mechanisms responsible for tumor escape from host immune surveillance.

While pancreatic cancer accounts for only 3% of all cancer in the US, it remains the fourth-leading cause of cancer related deaths in the US in 2015. The prevalence of pancreatic cancer is roughly equal between men and women. In 2015, approximately 48,960 people are estimated to be diagnosed with pancreatic cancer, and approximately 40,560 are expected to die from the disease. The relative five-year survival for patients with pancreatic cancer is 25% if the cancer is local at the time of diagnoses and only 2% if the cancer has metastasized at the time of diagnosis. New pharmaceutical compositions and methods of preventing and treating cancers must be developed to enhance the quality and span of life of subjects inflicted with cancer.

### SUMMARY OF THE INVENTION

One embodiment of the present invention is a method of preventing or treating cancer in a subject comprising: administering a vaccine to the subject; administering an agent that inhibits indoleamine 2,3-dioxygenase to the subject; and thereby treating or preventing the cancer in the subject. The present invention may treat or prevent any cancer including gastrointestinal, pancreatic, and pancreatic ductal adenocarcinoma (PDA) as examples. One or more embodiments of the present invention result in increased infiltration of CD8+T lymphocytes, activated CD8+ T cells, and interferon gamma (IFNy) producing CD8+ T cells into PDA tumor microenvironment (TME).

Suitable agents that inhibit indoleamine 2,3-dioxygenase may include and anti-indoleamine 2,3-dioxygenase antibody; an agent that inhibits indoleamine 2,3 dioxygenase gene expression; an agent that inhibits indoleamine 2,3-dioxygenase-1, indoleamine 2,3-dioxygenase-2, or a combination thereof; and an agent selected from the group Norhamane, Rosmarinic acid, COX-2, a combination thereof, or a pharmaceutically acceptable salt, solvate, or stereoisomer thereof, as examples. An example of a suitable agent that is a chemical inhibitor of idoleamine 2,3-dioxygenase includes a pharmaceutical composition comprising a compound of Formula I′, a compound of Formula I″, or a mixture thereof:

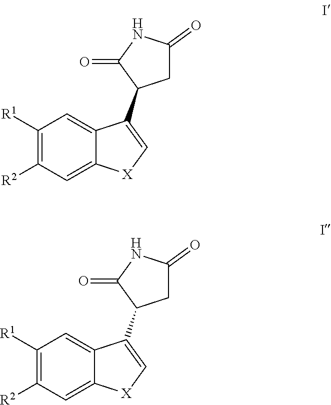

or a pharmaceutically acceptable enantiomer, salt, solvate or prodrug thereof, wherein:

X represents —NH— or —CQ²=CQ³-;

Q² and Q³ each independently represent H or C₁ to C₆ alkyl;

R1 and R2 each independently represent H, halo, cyano, C1 to C6 alkyl or C1 to C6 alkoxy;

and at least one pharmaceutically acceptable carrier.

The agent that inhibits indoleamine 2,3-dioxygenase and/or the vaccine is/are preferably administered in an amount that: decreases oxidoreductase activity of indoleamine 2,3-dioxygenase in the subject compared to a reference subject that is not administered the agent that inhibits indoleamine 2,3-dioxygenase-1 and/or the vaccine; that decreases oxidoreductase activity of indoleamine 2,3-dioxygenase in cancer cells of the subject compared to when the reference subject is not administered the inhibitor of indoleamine 2,3-dioxygenase and/or vaccine; that enhances immune system recognition of tumor associated antigens in the subject compared to when the reference subject is not administered the agent that inhibits idoleamine 2,3-dioxygenase and/or the vaccine; that inhibit cancer growth in the subject when compared to a reference subject not administered the agent that inhibits of indoleamine 2,3-dioxygenase and/or the vaccine, as examples. The reference subject may be a second subject or the subject (patient) who has not received the agent that inhibits idoleamine 2,3-dioxygenase before or after treatment with the agent.

The methods or the present invention may also administer to a subject a second agent such as an immune modulating dose of cyclophosphamide, wherein the immune modulating dose of cyclophosphamide is 100 mg/kg, as an example. Additional agents include anti PD-1, anti PD-L1, or a combination thereof. Any of the agents and/or vaccines of the present invention are preferably administered intraperitoneally or orally. It is also suitable for the vaccine and the agent that inhibits indoleamene 2,3-dioxygenase to be administered simultaneously.

A suitable vaccine includes an allogeneic PDA tumor cell engineered to secrete granulocyte macrophage colony-stimulating factor (GM-CSF) or a vaccine that comprises GM-CSF-secreting PDA vaccine; a Listeria monocytogene (LM)-based vaccine, preferable where the Listeria monocytogens (Lm)-based vaccine expresses and Annexin A2 (ANXA2) antigen, as examples.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function such as indoleamine 2,3-dioxygenase (an oxidoreductase) catalyzing the degradation of the essential amino acid tryptophan (trp) to N-formyl-kynurenine.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind, for example, PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “anti-Indoleamine 2,3-dioxygenase antibody” is meant an antibody that selectively binds a Indoleamine 2,3-dioygenase.

By “disease” is meant any condition or disorder that damages or interferes with the normal function of a cell, tissue, or organ. Examples of diseases include pancreatic cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “express” refers to the ability of a gene to express the gene product including for example its corresponding mRNA or protein sequence (s).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “GVAX” refers to a cancer vaccine composed of whole tumor cells genetically modified to secret the immune stimulatory cytokine, granulocyte-macrophage colony-stimulating factor (GM-CSF), and then irradiated to prevent further cell division. The product exists as both autologous (patient specific) and allogeneic (non-patient specific) therapy. The preferred GVAX used in the present invention is made from pancreatic cancer cells that have been genetically modified to produce the cytokine GM-CSF.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

“Immunoassay” is an assay that uses an antibody to specifically bind an antigen (e.g., a marker). The immunoassay is characterized by the use of specific binding properties of a particular antibody to isolate, target, and/or quantify the antigen.

The terms “Indoleamine 2,3-dioxygenase is protein including Indoleamine 2,3-dioxygenase 1 and Indoleamine 2,3-dioxygenase 2, as examples, encoded by the “IDO1” “IDO2” or “IDO” gene that functions as a heme-containing, monomeric oxidoreductase that catalyzes the degradation of the essential amino acid tryptophan (trp) to N-formyl-kynurenine, which can be subsequently metabolized through a series of steps to form nicotinamide adenine dinucleotide (NAD⁺). The Indoleamine-2,3-dioxygenase protein may also be referred to as IDO1, IDO2, or IDO.

The term, “obtaining” as in “obtaining an agent” includes synthesizing, purchasing, or otherwise acquiring the agent.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

The term “PD-1” refers to program death receptor-1. The term “anti PD-1 refers to one or more entity, such as an antibody for example, that bind to PD-1 and modulate its activity, preferably inhibit its activity. The term “PD-L1” refers to one of the major ligands of PD-1 called program death ligand 1. The term “anti PD-L1” refers to one or more entity, such as an antibody for example, that bind to PD-L1.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” is meant a negative alteration of at least 10%, 25%, 50%, 75%, or 100%.

A “reference” refers to a standard or control conditions such as a sample (human cells) or a subject that is a free, or substantially free, of an agent such as one or more inhibitors of Indoleamine 2,3-dioxygenase and/or a vaccine.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

As used herein, the term “sensitivity” is the percentage of subjects with a particular disease.

As used herein, the term “specificity” is the percentage of subjects correctly identified as having a particular disease i.e., normal or healthy subjects. For example, the specificity is calculated as the number of subjects with a particular disease as compared to non-cancer subjects (e.g., normal healthy subjects).

By “specifically binds” is meant a compound or antibody that recognizes and binds a polypeptide of the invention, but which does not substantially recognize and bind other molecules in a sample, for example, a biological sample, which naturally includes a polypeptide of the invention.

As used herein, the term “subject” is intended to refer to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for pancreatic cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

### BRIEF DESCRIPTION OF THE DRAWINGS

FIG. 1 illustrates the IDO1 expression observed in the tumor epithelium of pancreatic cancer. An anti-IDO1 antibody was used for staining.

FIG. 2 illustrates IDO1 expression in tumor epithelium among vaccinated vs. unvaccinated patients. An IDO1 antibody was used for staining.

FIG. 3 illustrates high IDO1 expression in tumor epithelium correlates with increased CD8+ infiltrates. An anti-IDO1 antibody was used for staining.

FIG. 4 illustrates inhibition of IDO combined with GVAX increases survival in liver metastasis mouse model. Survival Proportions: 90 Day survival of all groups.

FIG. 5 illustrates the combination of IDO inhibitor and vaccines therapy further enhances the infiltration of CD137+ or OX40+ T cells into pancreatic tumors of the mouse model.

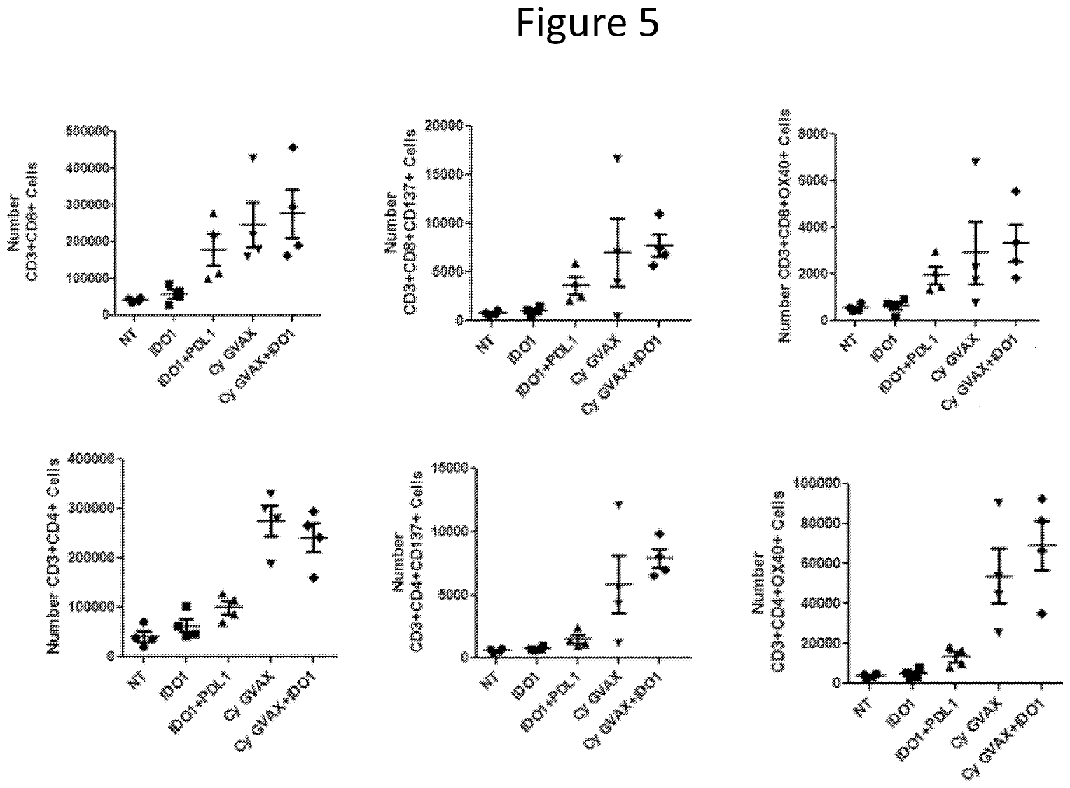

FIG. 6 illustrates inhibition of IDO combined with GVAX suppressed multiple immune checkpoint pathways (PD-1 and LAG-3).

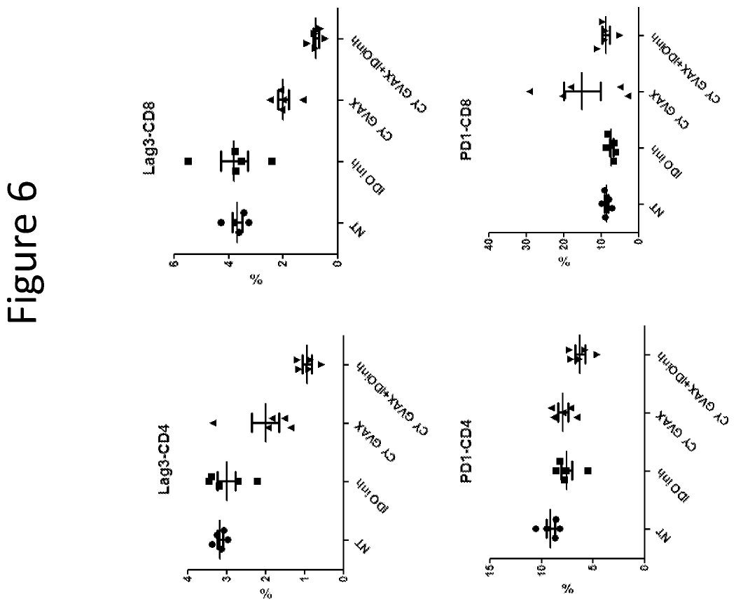

### DETAILED DESCRIPTION OF THE INVENTION

Examples of an indoleamine 2,3-dioxygenase inhibitors include compounds of Formula Ia:

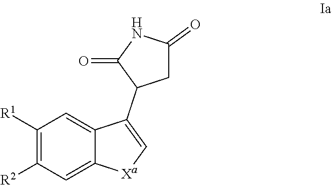

wherein X^{a} represents —NH— or —CQ²=CQ³-; Q2 and Q3 each independently represent H of C1 to C6 alkyl, preferably Q² and Q³ each independently represent H or methyl, more preferably Q² and Q³ represents H; R1 and R2 each independently represent H, halo, cyano, C1 to C6 alkyl or C1 to C6 alkoxy, preferably R1 and R2 each independently represent H or halo. Other inhibitors are further described in a patent application having an international publication number WO 2015/173764 A1 that is incorporated by reference such as a compound of Formula I′, a compound of Formula I″, or a mixture thereof:

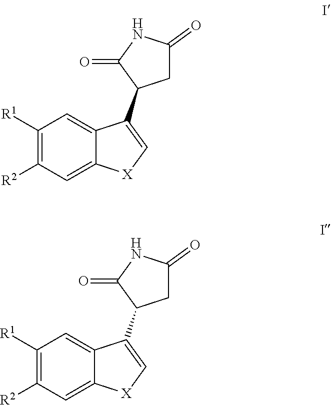

or a pharmaceutically acceptable enantiomer, salt, solvate or prodrug thereof, wherein:
X represents —NH— or —CQ²=CQ³-;
Q² and Q³ each independently represent H or C1 to C6 alkyl;
R1 and R2 each independently represent H, halo, cyano, C1 to C6 alkyl or C1 to C6 alkoxy;
   and at least one pharmaceutically acceptable carrier.

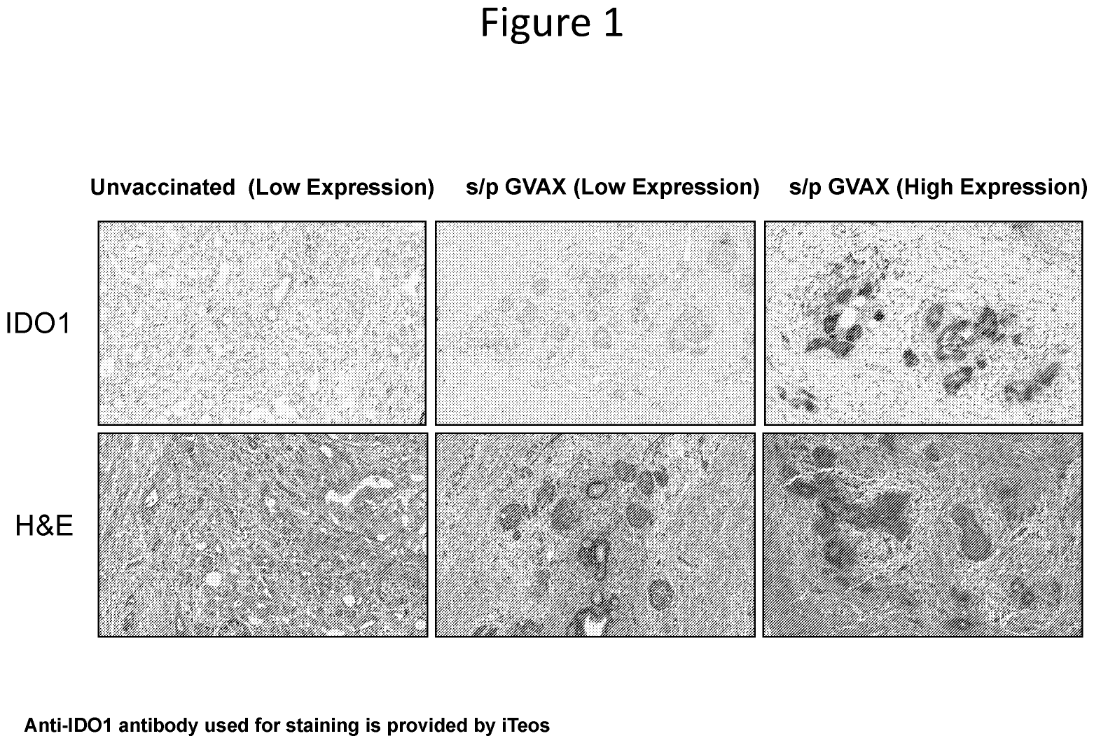

As further described in Example 1 and FIG. 1, the combination treatment regimen of cyclophosphamide, GVAX vaccine and oral IDO inhibitor, prolonged the life of mice in those groups. Other known IDO inhibitors include Norhamane, Rosmarinic acid and COX-2. The treatment with cyclophosphamide and GVAX vaccine also improved the survival of the mice, but the addition of the oral IDO1 inhibitor to this treatment further prolonged survival. The use of the IDO1 inhibitor without cyclophosphamide and GVAX vaccine had no significant effect on survival. The inventors also found that GVAX treatment induced IDO1 expression in both vaccine-induced lymphoid aggregates and tumor epithelium in human pancreatic adenocarcinoma. The inventors conclude: 1. The combination of the vaccine therapy (such as GVAX as an example) and IDO inhibitor is superior to either vaccine therapy or IDO inhibitor for pancreatic cancer treatment. 2. Vaccine therapy induced the expression of IDO in pancreatic adenocarcinoma, thus, primes the patients for IDO inhibitor treatment. 3. The combination of vaccine therapy and anti-PD-1/PD-1 therapy has been elsewhere shown to be superior to either anti-PD-1/PD-L1 therapy or vaccine therapy alone. Thus, the IDO inhibitor can be combined together with vaccine therapy and anti-PD-1 or PD-L1 therapy.

Embodiments of the disclosure concern methods and/or compositions for treating and/or preventing cancer, specifically pancreatic cancer in which modulation of IDO1 is directly or indirectly related. In certain embodiments, individuals with cancer such as pancreatic cancer are treated with a modulator of the IDO1, and in specific embodiments an individual with cancer is provided a modulator of IDO1, such as an inhibitor of its expression and/or activity in combination with GVAX.

In certain embodiments, the level to which an inhibitor of IDO1 activity and/or expression inhibits IDO1 activity and/or expression may be any level so long as it provides amelioration of at least one symptom of the cancer, including pancreatic cancer. The level of expression and/or activity may decrease by at least 2, 3, 4, 5, 10, 25, 50, 100, 1000, or more fold expression and/or activity compared to the level of expression and/or expression in a standard or reference, in at least some cases. An individual may monitor expression levels of IDO1 expression using standard methods in the art, such as northern assays or quantitative PCR, for example. An individual may monitor activity levels of IDO1 activity using standard methods in the art.

An individual known to have cancer, suspected of having cancer, or at risk for having cancer may be provided an effective amount of an inhibitor of IDO1 expression and GVAX. Those at risk for cancer may be those individuals having one or more genetic factors, may be of advancing age, and/or may have a family history, for example.

In particular embodiments of the disclosure, an individual is given an agent, for cancer therapy in addition to the one or more inhibitors of IDO1. Such additional therapy may include GVAX, for example. When combination therapy is employed with one or more inhibitor of IDO1, the additional therapy may be given prior to, at the same time as, and/or subsequent to the inhibitor of IDO1.

### Pharmaceutical Preparations

Pharmaceutical compositions of the present invention comprise an effective amount of one or more inhibitors of expression and/or activity of IDO1, as well as one or more agent such as GVAX, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one inhibitor of expression and/or activity of IDO1 or additional active ingredient such as GVAX will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

A composition including an inhibitor of expression and/or activity of IDO1, GVAX, or the combination thereof may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

GVAX and the inhibitor of expression and/or activity of IDO1 may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include inducer of expression of PGC-1α, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, GVAX and the inducer of expression and/or activity of IDO1 may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In one embodiment of the present disclosure, GVAX and an inhibitor of expression and/or activity of IDO1 are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, GVAX and inhibitors of expression and/or activity of IDO1 may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other preferred embodiments of the invention, the active compounds including GVAX and an inhibitor of expression and or activity of IDO1 may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compositions described herein may be comprised in a kit. In a non-limiting example, GVAX and an inhibitor of expression and/or activity of IDO1 (for example), Norhamane, Rosmarinic acid and COX-2) may be comprised in a kit.

The kits may comprise a suitably aliquoted GVAX, inhibitor of expression and or activity of IDO1, or both, and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing GVAX, inhibitor of expression and/or activity of IDO1 and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. GVAX, inhibitor of expression and/or activity of IDO1, or a combination thereof, composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

The inventors performed hemi-splenectomy with the injection of 2×10⁶ Panc02 murine pancreatic adenocarcinoma cells on C57/B16 mice, aged 9-10 weeks to model metastasis of pancreatic tumor cells to the liver. Mice were divided into treatment groups and dosed with cyclophosphamide and GVAX vaccine as well as an oral IDO1 inhibitor PF-06840003, according to a 28-day dosing scheme.

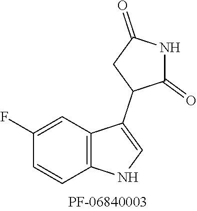 [SMILES: O=C1CC(c2c[nH]c3ccc(F)cc23)C(=O)N1]

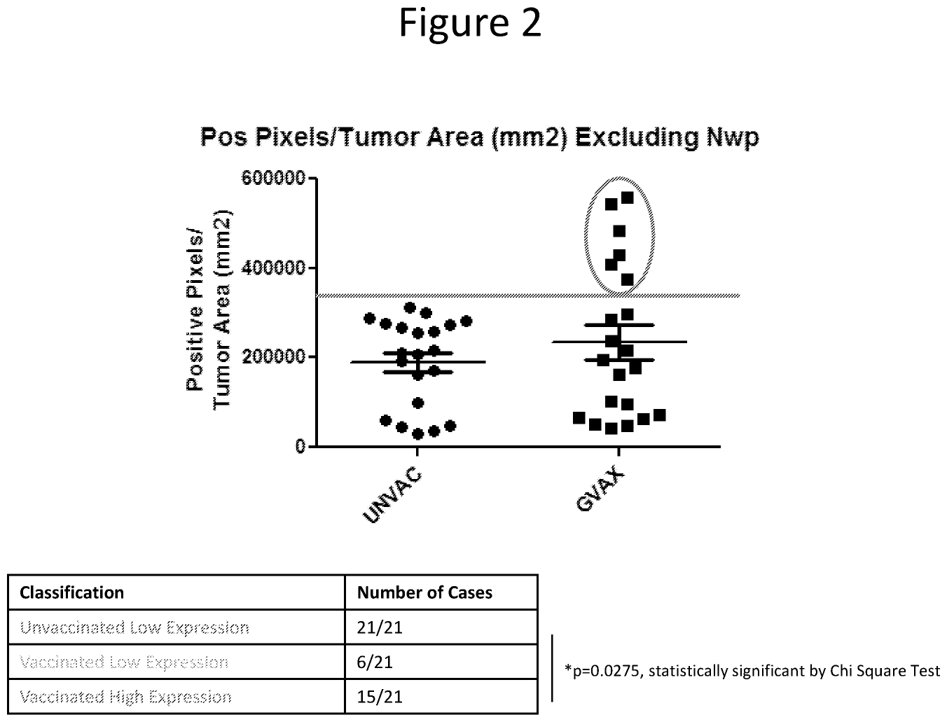

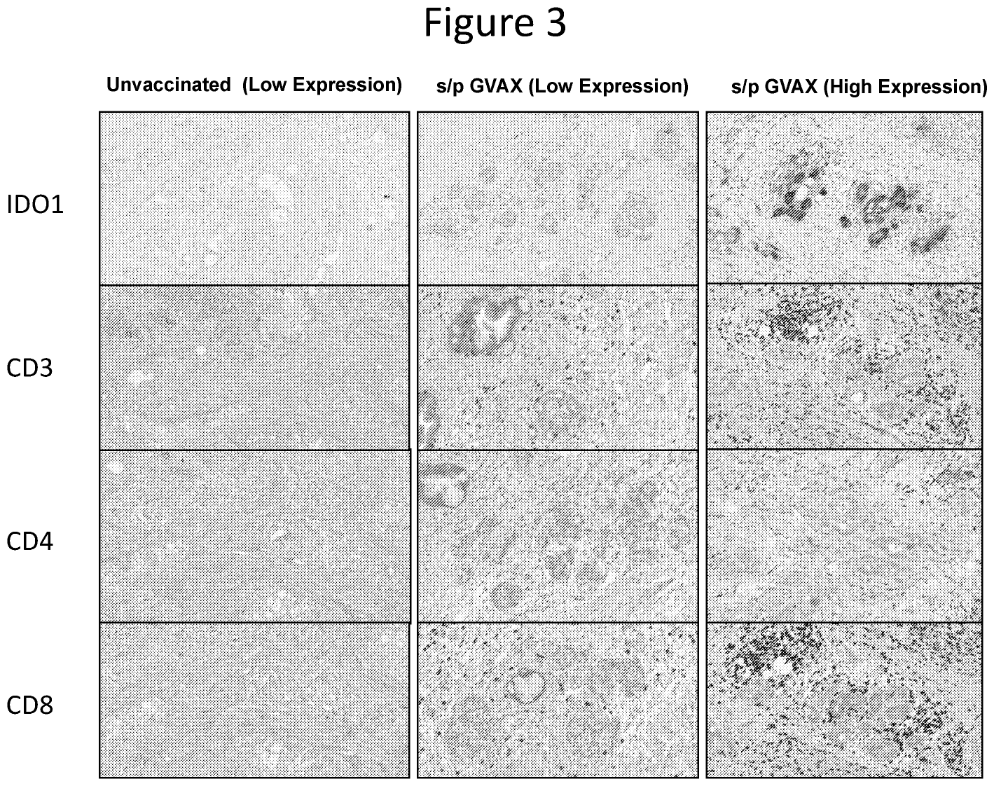

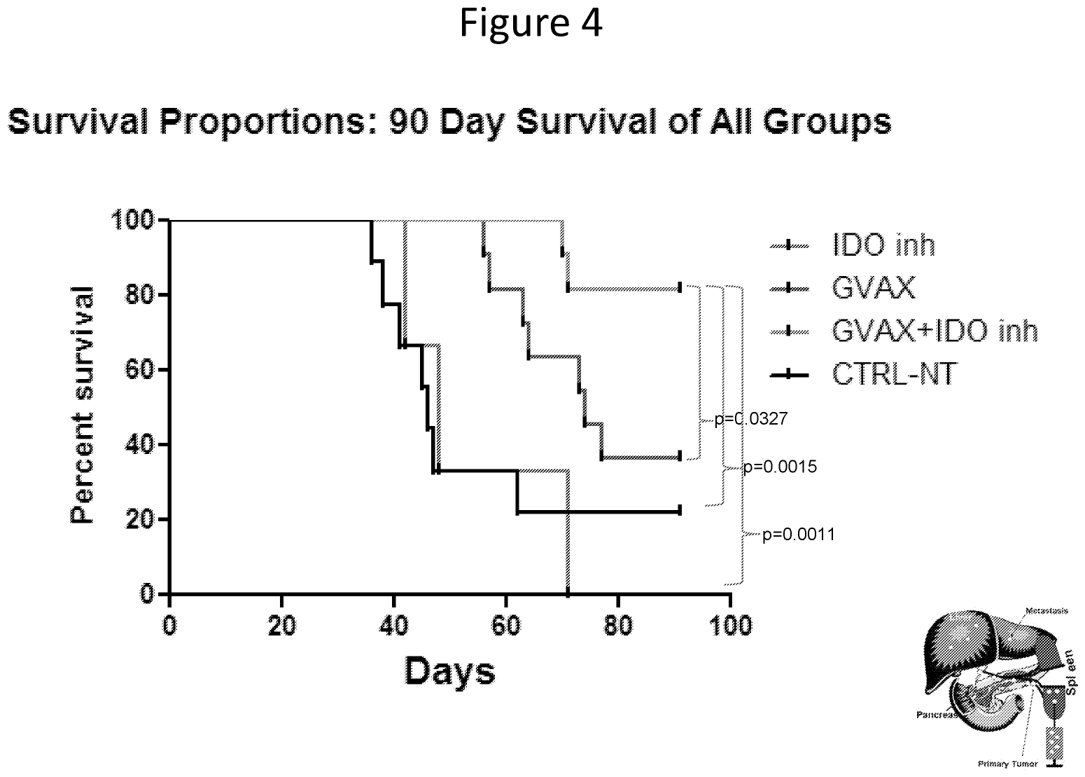

An oral methocel vehicle was given as a placebo treatment. The IDO inhibitor PF-06840003 and oral methocel compounds were administered twice daily by oral gavage, starting on post-operative day 3 and extending through post-operative day 28. Cyclophosphamide was administered on post-operative day 3. GVAX vaccine was administered on post-operative days 4, 7, 14 and 21. Mice were followed for survival for 90 days. Mice exhibiting severe clinical symptoms were humanely euthanized as per standard animal care and use protocol. Surviving mice following the 90 day period were humanely euthanized (FIG. 4). The inventors also performed the immunohistochemistry analysis on the pancreatic adenocarcinoma resected from patients who received the GVAX treatment by using anti-IDO1 antibody supplied by iTeos (FIGS. 1-3).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preventing or treating pancreatic cancer comprising:
administering a vaccine to the subject;
administering an agent that inhibits indoleamine 2,3-dioxygenase to the subject;
thereby treating or preventing the pancreatic cancer in the subject.

2-3. (canceled)

4. The method of claim 3, wherein said pancreatic cancer is pancreatic ductal adenocarcinoma (PDA).

5. The method of claim 1, wherein the vaccine comprises and allogeneic PDA tumor cell engineered to secrete granulocyte macrophage colony-stimulating factor (GM-CSF).

6. The method of claim 5, wherein the vaccine comprises GM-CSF-secreting PDA vaccine.

7. The method of claim 4, wherein infiltration of CD8+T lymphocytes, activated CD8+ T cells, and interferon gamma (IFNy) producing CD8+ T cells into PDA tumor microenvironment (TME) is increased.

8. The method of claim 1, wherein the agent that inhibits indoleamine 2,3-dioxygenase comprises and anti-indoleamine 2.3-dioxygenase antibody.

9. The method of claim 1, wherein the agent that inhibits idoleamine 2,3-dioxygenase comprises a pharmaceutical composition comprising a compound of Formula I′, a compound of Formula I″, or a mixture thereof: 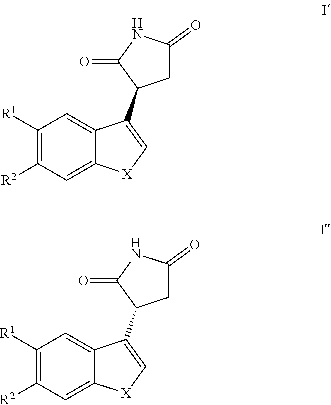
or a pharmaceutically acceptable enantiomer, salt, solvate or prodrug thereof, wherein:
X represents —NH— or —CQ²=CQ³-;
Q² and Q³ each independently represent H or C₁ to C6 alkyl;
R1 and R2 each independently represent H, halo, cyano, C1 to C6 alkyl or C1 to C6 alkoxy;
and at least one pharmaceutically acceptable carrier.

10. The method of claim 1, wherein the agent that inhibits indoleamine 2,3-dioxygenase inhibits indoleamine 2,3-dioxygenase gene expression.

11. The method of claim 1, wherein the agent that inhibits indoleamine 2,3-dioxygenase inhibits indoleamine 2,3-dioxygenase-1.

12. The method of claim 1, wherein the agent that inhibits indoleamine 2,3-dioxygenase is selected from the group Norhamane, Rosmarinic acid,COX-2, a combination thereof, or a pharmaceutically acceptable salt, solvate, or stereoisomer thereof.

13. The method of claim 1 further comprising administering to the subject an immune modulating dose of cyclophosphamide.

14. The method of claim 13 wherein the immune modulating dose of cyclophosphamide is 100 mg/kg.

15. The method of claim 14, wherein the cyclophosphaminde is administered intraperitoneally or orally.

16. The method of claim 1 further comprising administering a second agent selected from the group comprising anti PD-1, anti PD-L1, or a combination thereof.

17. The method of claim 1, wherein the agent that inhibits indoleamine 2,3-dioxygenase is administered in an amount that decreases oxidoreductase activity of indoleamine 2,3-dioxygenase in the subject compared to a reference subject that is not administered the agent that inhibits indoleamine 2,3-dioxygenase.

18. The method of claim 1, wherein the agent that inhibits indoleamine 2,3-dioxygenase is administered in an amount that decreases oxidoreductase activity of indoleamine 2,3-dioxygenase in cancer cells of the subject compared to when the reference subject is not administered the inhibitor of indoleamine 2,3-dioxygenase.

19. The method of claim 1, wherein the agent that inhibits idoleamine 2,3-dioxygenase is administered in an amount that enhances immune system recognition of tumor associated antigens in the subject compared to when the reference subject is not administered the agent that inhibits idoleamine 2,3-dioxygenase.

20. The method of claim 1 wherein inhibitor of indoleamine 2,3-dioxygenase and the vaccine inhibit cancer growth in the subject when compared to when a reference subject not administered the agent that inhibits of indoleamine 2,3-dioxygenase and/or the vaccine.

21. The method of claim 1, wherein the vaccine comprises a Listeria monocytogene (LM)-based vaccine.

22. The method of claim 23 wherein the Listeria monocytogens (Lm)-based vaccine expresses and Annexin A2 (ANXA2) antigen.

23. The method of claim 1 wherein the agent that inhibits Indoleamine 2,3-dioxygenase is selected from the group consisting of an inhibitor of indoleamine 2,3-dioxygenase-1, indoleamine 2,3-dioxygenase-2, or a combination thereof.
